(19) United States
(12) Patent Application Publication
(10) Pub. No.: US 2026/0013773 A1
(43) Pub. Date: Jan. 15, 2026

(54) INTRACRANIAL IMPLANT SYSTEM WITH DATA SENSING

(71) Applicant: NEUROINSIGHTS, INC., Keego Harbor, MI (US)

(72) Inventors: Daniel Abrams, Denver, CO (US); Thomas Prentice, Winter Haven, FL (US)

(21) Appl. No.: 18/771,398

(22) Filed: Jul. 12, 2024

(51) Int. Cl. A61B 5/293; A61B 5/00

(52) U.S. Cl. CPC A61B 5/293; A61B 5/6814; A61B 5/686; A61B 2560/0406; A61B 2560/0468; A61B 2562/227

(57) ABSTRACT

An intracranial implant sensing device includes a flange receivable within a burr hole formed in a human skull and defining an elongated slot. The device further includes an elongate body having an arcuate sidewall defining an open guide for a ventricular catheter. The elongate body is connected to the flange such that a portion of the sidewall is disposed within or at a perimeter of the slot. A sensing electrode of the device is connected to the sidewall of the elongate body.

### TECHNICAL FIELD

This disclosure relates to an intracranial implant system for sensing, collecting, and storing intracranial electroencephalogram data.

### SUMMARY

According to one embodiment an intracranial implant sensing device includes a flange receivable within a burr hole formed in a human skull and defining an elongated slot. The device further includes an elongate body having an arcuate sidewall defining an open guide for a ventricular catheter. The elongate body is connected to the flange such that a portion of the sidewall is disposed within or at a perimeter of the slot. A sensing electrode of the device is connected to the sidewall of the elongate body.

The intracranial implant sensing device may further include a riser extending upwardly from the flange and an anchor plate extending radially outward from an upper end of the riser and restable on the human skull. A lower end of the riser may be connected to the arcuate sidewall. The riser, the anchor plate, and the flange may be integrally formed or separately formed.

The flange may have a top and an arcuate sidewall depending from the top and extending circumferentially less than 350 degrees to define an open end of the elongated slot. In some embodiments, the flange may extend between 180 and 270 degrees.

The sensing electrode is configured to sense intracranial electroencephalogram signals. The sensing electrode may be a plurality of sensing electrodes. For example, the device may further include a second sensing electrode connected to the sidewall of the elongate body and longitudinally spaced from the first sensing electrode. Wires are connected to the one or more electrodes. For example, a wire has a first end connected to the sensing electrode and extending through the riser and anchor plate to a second end. In addition to a sensing electrode(s), the device may further include a ground electrode. The ground electrode may at least partially surround the elongate body.

The elongate body may be removably connected to the flange by features. For example, clips, detents, or the like may connect the elongate body to the flange. The elongate body may be received within the slot and removably connected to the flange by features provided in the slot.

The sidewall of the elongate body may circumferentially extend between 90 and 270 degrees, inclusive. The open side of the guide may be circumferentially aligned with the open end of the slot.

The intracranial implant device may also include a coil connected to a second end of the wire and configured to inductively couple with a second coil.

According to another embodiment, a medical device includes a sensing device having an intracranial implant with a burr-hole flange receivable within a burr hole formed in a human skull and defining an elongated slot. An anchor plate is fixed to the implant such that the anchor plate is spaced above the flange and is configured to rest on the human skull. A sensor stem is joined to the flange and is insertable into a human brain. At least one sensing electrode is disposed on the stem. A first electronic device is connected to the electrode and implantable subcutaneously. A second electronic device is receivable by the first electronic device to electrically couple with the first electronic device and receive data collected by the electrode. A catheter is receivable within the stem. The catheter may be associated with an apparatus configured to drain cerebrospinal fluid from the brain, e.g., a ventriculoperitoneal shunt.

The stem may have a curved sidewall that defines an open guide configured to receive the catheter. The elongate slot may be an open slot having an entrance defined between opposing walls of the flange. The stem may have a partial sidewall with an open side that is aligned with the entrance of the open slot.

The implant of the medical device may further have a riser connected between the anchor plate and the flange. At least one of the sensing electrodes of the medical device may have at least two sensing electrodes.

The flange of the medical device may be horseshoe shaped. The sensing device may have a matching horseshoe-shaped ground electrode disposed on a bottom of the flange.

According to yet another embodiment, a system for collecting and sending intracranial electroencephalogram data includes an implant having a burr-hole flange defining a first open slot, an anchor plate located above a top of the flange, a riser connected between the flange and the anchor plate, and an elongate body extending downwardly from the flange. The elongate body has a partial arcuate sidewall defining an open guide. A sensing electrode is connected to the elongate body. A first device is configured to receive data from the electrode, and a second device is electrically coupled to the first device. A storage device is electrically coupled to the second device and configured to store the data.

### BRIEF DESCRIPTION OF THE DRAWINGS

FIG. 1 is a front perspective view of an intracranial implant sensing device.

FIG. 2 is a bottom view of the intracranial implant sensing device.

FIG. 3 is a back perspective view of the intracranial implant sensing device.

FIG. 4 is a cross-sectional perspective view showing the intracranial implant sensing device and a ventriculoperitoneal shunt implanted in a human brain.

FIG. 5 is a perspective view of an intracranial implant system showing subcutaneous components thereof including the intracranial implant sensing device.

FIG. 6 is a perspective view of the intracranial implant system showing external components thereof.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the present invention. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis 10 (also known as a centerline) is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made. The terms, connected, attached, etc., refer to directly or indirectly connected, attached, etc., unless otherwise indicated explicitly or by context.

The ability to monitor brain activity in patients implanted with an external ventricular drain (EVD) or a ventriculoperitoneal (VP) shunt is limited. Existing methods for monitoring brain activity, such as magnetic resonance imaging (MRI), functional MRI (fMRI), surface-mounted electroencephalogram (EEG), and magnetoencephalogram (MEG) recordings, as well as other imaging techniques, are unable to continuously measure high-quality temporally relevant brain data over long periods of time. The below-described devices and systems provide a solution to this problem by providing intracranial sensing electrodes.

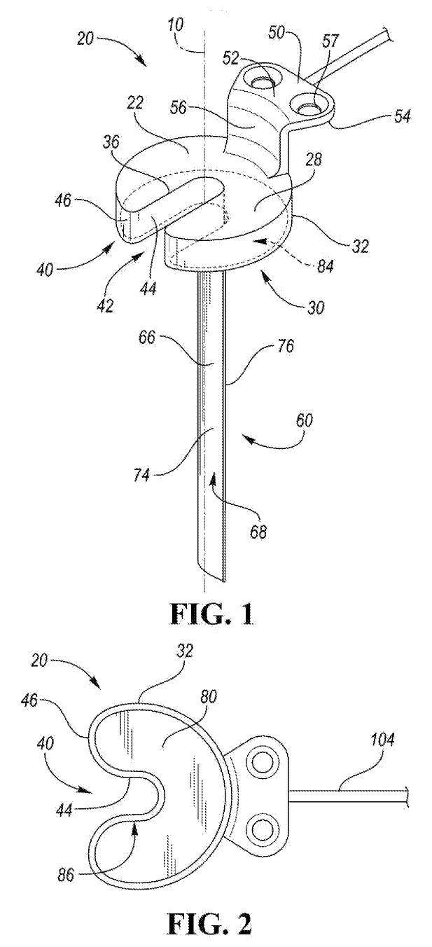

Referring to FIGS. 1–4, an intracranial implant sensing device 20 may include a burr-hole flange 22 receivable within a burr hole 24 of a human skull 26. The flange 22 may provide centering and support functionalities. The flange 22 includes a top 28, a bottom 30, and an arcuate sidewall 32 extending downwardly from the top 28 to the bottom 30. The flange 22 is sized to fit within the burr hole 24. When received within the burr hole 24, the sidewall 32 is in close proximity with the wall 34 of the burr hole and the top 28 is recessed from the top of the skull 26. In the illustrated embodiment, the flange 22 is circular, e.g., disc-shaped, albeit not complete. That is, the sidewall 32 does not extend 360 degrees around the central axis 10. Instead, the flange 22 includes an open front side 40 formed by an elongated slot 36. The slot 36 extends radially inward from an entrance 42 towards, and sometimes passed, the center point of the flange 22, which is located on the central axis 10. Thus, the flange 22 is horseshoe shaped in the illustrated example. The slot 36 includes one or more sidewalls 44 extending downwardly from the top 28. The sidewalls 44 may directly intersect with the sidewall 32 at the entrance 42 or chamfered or filleted walls 46 may be provided as a transition between the sidewalls 44 and the sidewall 32 as shown in the illustrated embodiment. In one or more embodiments, sidewall 32 extends 180 to 355 degrees, inclusive, around the flange 22. In other embodiments, the flange may be less than a half disc and have a sidewall that extends between 90 and 179 degrees. While the illustrated embodiment includes an open slot extending radially inward from a periphery of the flange, in other embodiments, the flange may be a complete disc with a closed slot defined within the circular periphery of the flange.

An anchor plate 50 secures the implant 20 to the skull 26. The anchor plate 50 is planar having a top 52 and a bottom 54 forming the major surfaces of the plate. The anchor plate 50 defines one or more fasteners holes 57 configured to receive a fastener that is inserted into the skull, e.g., a screw. In the illustrated embodiment, the anchor plate 50 includes two fastener holes 57. More or fewer fasteners and holes may be used in other embodiments. The anchor plate 50 is spaced above the top 28 of the flange 22 and at least partially circumferentially outboard of the flange 22 so that the bottom 54 is disposed on top of the skull 26. A riser 56 may be used to connect the anchor plate 50 to the flange 22. The riser 56 extends downwardly from the inner side of anchor plate 50 to the flange 22. The riser 56 may be attached to the top 28 of the flange 22, attached to the sidewall 32, or both. In one or more embodiments, the riser 56 and the anchor plate 50 may be integrally formed with each other. Additionally, the anchor plate 50, the riser 56, and the flange 22 may also be integrally formed with each other. Here, the base of the riser 56 may be integrated with the top 28 and sidewall 32 of the flange 22.

The anchor plate 50 and the riser 56 are minimally sized to provide space within and around the burr hole 24 for other components. For example, the anchor plate 50 and the riser 56 may extend less than 180 degrees circumferentially about the axis 10. In one or more embodiments, the riser 56 and/or the anchor plate 50 extends less than 50 degrees, 60 degrees, 70 degrees, 80, degrees, 90 degrees, 100 degrees, 110 degrees, or 120 degrees circumferentially about the axis 10. The thickness of the anchor plate 50 and the riser 56 are also minimized. For example, the anchor plate 50 is designed to be inserted below the skin of the patient and may have a thickness between 1 to 7 mm. The thickness of the riser 56 may be between 0.3 to 3 mm to optimize clearance within the burr hole 24 for other components including a base of a VP shunt reservoir.

An elongate body 60 (a stem) extends downwardly from the flange 22 and is configured to be inserted in the grey or white matter of the brain 61 in the direction of the lateral ventricle. The elongate body 60 may serve dual purposes. The first purpose may be to support a VP shunt 70, and a second purpose may be to support electrodes that sense brain activity. For example, the electrodes may be used to collect intracranial electroencephalogram (iEEG) data.

The elongate body 60 includes an upper end 62 connected with the flange 22 and a lower end 64. The lower end 64 may include a sharpened edge to facilitate insertion into the brain 61. The elongate body 60 may be removably or non-removably connected to the flange 22. The elongate body 60 includes an arcuate sidewall 66 defining a guide 68 for a ventricular catheter 72 of the VP shunt 70. The elongate body 60 may be a partial tube with the sidewall 66 extending axially relative to the centerline 10 and radially about the centerline 10. The sidewall 66 includes an inner circumferential side 74 that forms a surface of the guide 68 and an outer circumferential surface 76. In the illustrated embodiment, the sidewall 66 partially extends around the centerline 10 thus defining an open guide 68, e.g., an open cavity. The partial sidewall 66 has a smaller footprint than a complete sidewall thus reducing disturbance of brain tissue. The partial sidewall 66 also increases clearance for placement of additional monitors and for easy placement and replacement of a catheter with a wide variety of other catheters. The sidewall 66 may extend circumferentially between 60 and 270 degrees about the centerline 10. The sidewall 66 is also thin, e.g., between 0.1 to 1 mm.

An inner diameter of the sidewall, i.e., the diameter that forms the inner side 74, may correspond with the width of the elongate slot 36, e.g., they are the same or substantially the same. Depending on the amount of circumferential rotation of the elongate body 60, the sidewall 66 may define a longitudinally extending slot forming an open side of the guide 68. In other embodiments, the elongate body 60 may be a tube that defines a closed guide e.g., a lumen, that extends longitudinally through the center of the tube.

The elongate body 60 may be connected to the flange 22 such that the guide 68 is aligned with the elongate slot 36. This allows the catheter 72 to be inserted within the device 20 from the top. In one embodiment, the inner surface 74 of the sidewall 66 is flush with a sidewall 44 (such as the back wall opposite the entrance 42) of the slot 36. In other embodiments, the elongate body 60 is received within the elongate slot 36. Here, the outer surface 76 may be disposed against a sidewall(s) 44 of the slot 36.

In the illustrated embodiment, the elongated body 60 is concentric with the centerline 10 of the flange 22. In other embodiments, the elongated body my be eccentric with the flange 22. For example, in embodiments where the flange is less than a half disc, the elongated body may be eccentric.

As discussed above, the elongate body 60 may be removably or non-removably connected to the flange 22. In one or more embodiments, the elongate body 60 is integrally formed with the flange 22. In other embodiments, the elongate body may be non-removably connected to the flange 22 using adhesive, welding, or permanent fasteners. The elongate body 60 may be removably connected to the flange 22 through snap fit, interference fit, or removable fasteners. For example, the elongate slot 36 may include features that cooperate with the upper end of the elongate body when the elongate body 60 is received within the slot 36. Example features of the slot 36 include detents or clips that engage with the sidewall 66. In that example, the elongate body 60 snap fits to the flange 22 when the elongate body 60 fully seats within the slot 36. Alternatively, the width of the slot 36 may be narrower than the diameter of the sidewall 66 to create an interference fit between the elongate body 60 and the flange 22.

One or more electrodes are attached to the device 20. In the illustrated embodiment, the device 20 includes a ground electrode 80 and one or more sensing electrodes 82. The ground electrode 80 may be placed on the device 20 anywhere the electrode 80 will contact the brain 61 of the patient. In one embodiment, the ground electrode 80 is disposed on the bottom 30 of the flange 22. The flange 22 may define an open cavity 84 on the bottom 30 that receives the ground electrode 80 therein. Here, the ground electrode 80 has a shape that matches the shape of the flange, albeit smaller to fit within the cavity 84 defined by the sidewall 32. In the illustrated embodiment, the ground electrode 80 is also horseshoe shaped and defines an open area 86 between the prongs of the horseshoe providing clearance for the elongate body 60 and the VP shunt 70. In other embodiments, the ground electrode 80 may be much smaller than the flange 22 and may be a disk-shaped electrode received on the bottom 30 of the flange at a location away from the slot 36 so that it does not interfere with the elongate body 60, the VP shunt 70, or the like.

At least one sensing electrode 82 may be attached to the sidewall 66 of the elongate body 60 to be placed within the white or grey matter of brain 61 when the device 20 is implanted. For example, first and second electrodes 90 and 92 may be supported on the elongate body 60. The first electrode 90 may be an upper electrode and the second electrode 92 may be a lower electrode. The electrodes 90 and 92 may be disposed on the outer side 76 of the sidewall 66. Placing the electrodes 90, 92 on the outer side 76 eliminates interference with the VP shunt 70. Additional sensing electrodes may be placed on the sidewall 66 and may also be on the outer side 76. The electrodes may be axially arranged one after the other in the longitudinal direction of the elongate body 60 with equal or unequal spacing therebetween.

The electrodes 90, 92 are intracranial electrodes that may be placed within a cerebral ventricle (“intraventricular”) of the brain 61. The electrodes 90, 92 are configured to sense iEEG signals. iEEG signals include signals obtained from the electrodes 90, 92 positioned on the surface of the brain of a subject and within the brain of the subject, including the electrodes placed intraparenchymally, intracerebrally, intraventricularly, and intravascularly. While the illustrated embodiment includes two sensing electrodes, the device 20 may include additional sensing electrodes disposed along the elongate body 60. An additional electrode (or electrodes) that is external to the brain may also be provided on the device 20. This additional electrode(s) may be placed on a surface of the brain or in contact with the skull depending upon their purpose.

Each electrode 90, 92 may include a planar body 93 of metal such as platinum, platinum-iridium alloy, or gold, for example. The planar body 93 may be solid metal or may include a substrate with a coating of sensing metal. For example, platinum, platinum-iridium alloy, or gold may be sputtered (or otherwise applied) over a substrate, such as another metal.

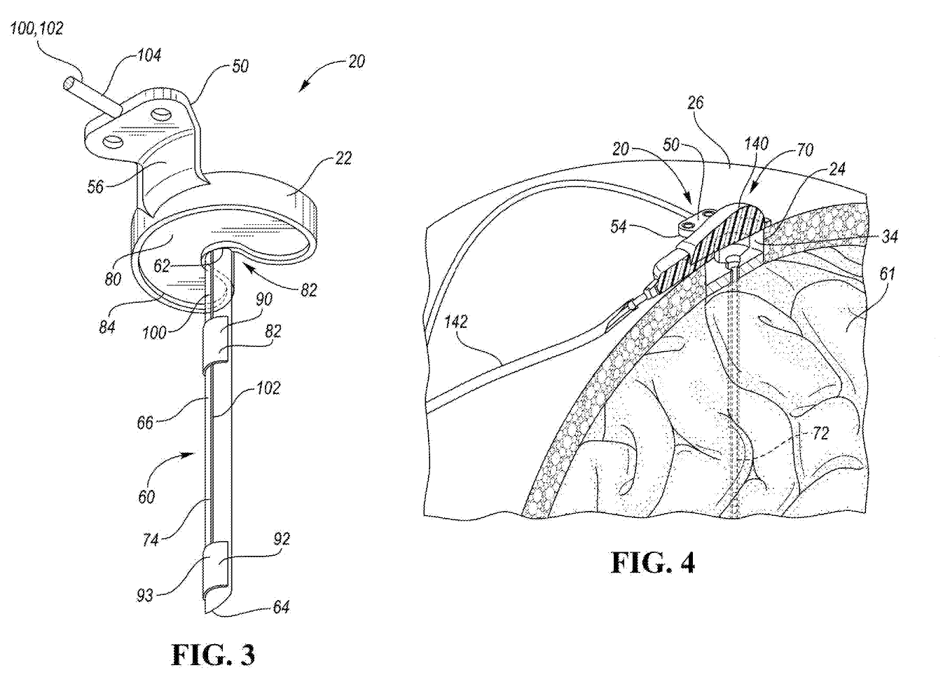

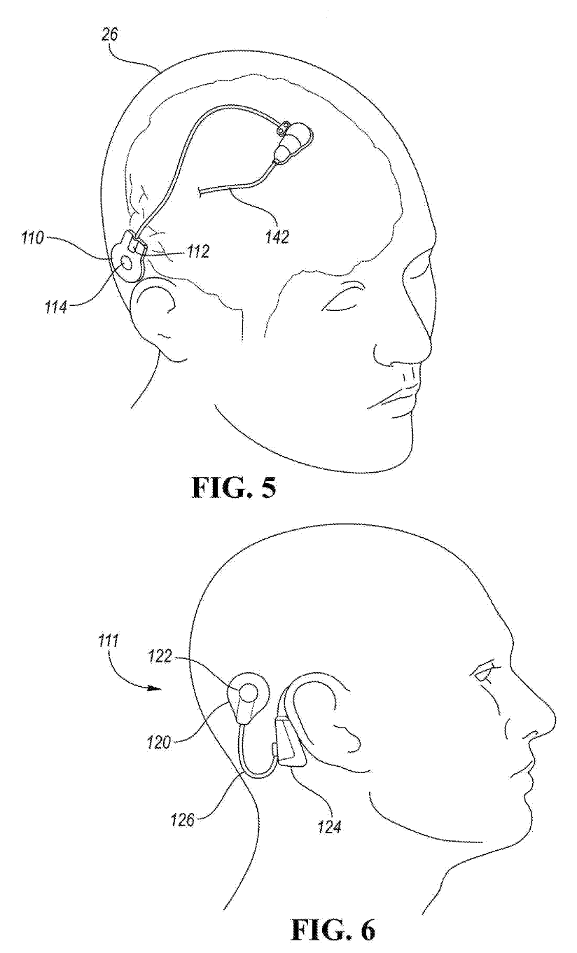

Referring to FIGS. 3, 5, and 6, each of the electrodes 90, 92 may have an associated electrical conductor, e.g., a wire, extending from the electrode. For example, a first wire 100 is connected to the electrode 90 and a second wire 102 is connected to the electrode 92. The wires may be connected to the electrodes by laser welding or other suitable joining techniques. The wires may be disposed on the surface 74 of the elongate body 60 or may be disposed internally within the elongate body 60. The wires 100, 102 are disposed on or located within the flange 22, the riser 56, and the anchor plate 50. The wires 100, 102 may be bundled into a wiring harness 104. The ground electrode 80 may also include an associated wire (not shown) that can be included in the wiring harness 104. The wiring harness 104 is designed to be placed subcutaneously.

The implant 20 may be formed of plastic, biocompatible metal (e.g., stainless steel, titanium, and nitinol), or other suitable material. In one or more embodiments, the implant 20 is a molded plastic in which many of the above-described components are integrally formed. In this embodiment, the wires 100, 102, electrodes 90, 92, and/or wiring harness 104 are over molded to be within the monolithic structure. This allows the wires to extend from the electrodes, through the elongate body 60, through the flange 22, up the riser 56, and out through the anchor plate 50 as shown in the example figures. Alternatively, the electrodes 90, 92 and wires 100, 102 may be on a surface of the implant 20. The surface may include grooves and recessed areas for receiving the wires and electrodes to streamline the implant 20.

The wiring harness 104 electrically connects the electrodes, e.g., sensing electrodes 90, 92, to an implanted electronics device 110 of a data processing and storage system 111. The device 110 is designed to be placed on the skull 26 below the skin of the patient. The device 110 may include a chip 112 and an implanted coil 114. The chip 112 may modulate signals, coordinates timing, and reset external wire manage electronic recording over time.

An external electronics device 120 is configured to electrically couple with the implanted device 110. The external device 120 is received on top of the skin of the patient and may be secured to the device 110 with magnets or other means, e.g., adhesive. For example, the internal device 110 may have a magnet that couples with a magnet withing the external device 120. The external device 120 is electrically connected with a wearable unit 124 that may be worn behind the ear, on the hip, or other location. A cord 126 may connect the wearable unit 124 with the external device 120. The wearable device 124 may include a controller, memory, storage, and a power source, e.g., a battery. For example, the controller may include a microprocessor or central processing unit (CPU) in communication with various types of computer-readable storage devices or media. Computer-readable storage devices or media may include volatile and nonvolatile storage in read-only memory (ROM), random-access memory (RAM), and keep-alive memory (KAM), for example. KAM is a persistent or non-volatile memory that may be used to store various operating variables while the CPU is powered down. Computer-readable storage devices or media may be implemented using any of a number of known memory devices such as PROMs (programmable read-only memory), EPROMs (electrically PROM), EEPROMs (electrically erasable PROM), flash memory, or any other electric, magnetic, optical, or combination memory devices capable of storing data, some of which represent executable instructions, used by the controller in controlling the vehicle. The controller may communicate with various sensors via an input/output (I/O) interface that may be implemented as a single integrated interface that provides various raw data or signal conditioning, processing, and/or conversion, short-circuit protection, and the like. Alternatively, one or more dedicated hardware or firmware chips may be used to condition and process particular signals before being supplied to the CPU.

The device 120 includes an external coil 122 configured to inductively couple with the internal coil 114 to provide power from the wearable unit 124 to the electrodes, e.g., 90, 92 of the implant 20. The external coil 122 is also configured to receive data from the internal coil 114. For example, the sensing electrodes 90, 92 output raw data indicative of the sensed iEEG signals to the internal coil 114 and the external coil 120 receives the raw data from the internal coil 114. That data is then sent to the controller of the wearable unit 124 that processes the raw iEEG data and saves the processed data in local storage such as an SD card. In some embodiments, the wearable unit 124 may include a transmitter that outputs the processed data to a receiver of an external system and/or a port for a wired connection to the external system. In other embodiments, the data transfer may be optical. That is, data is optically transmitted through scalp.

Referring to FIGS. 4 and 6, as discussed above, the sensing device 20 is designed to operate in conjunction with a VP shunt 70. The VP shunt 70 includes a ventricular catheter 72 that is inserted into the device 20 from the top via the slot 36. The catheter 72 may extend through the slot 36 and the guide 68 as it is supported by the elongate body 60 during inserting into the brain. A radius of the catheter 72 may be less than the radius of the sidewall 66 so that the catheter 72 may freely extend within the guide 68. The catheter 72 may be longer than the elongate body 60 thus extending deeper into the brain than the device 20. The elongate body 60 may include a feature for retaining the shunt catheter 72 within the guide 68. This may be beneficial during insertion of the shunt catheter 72 into the brain. The feature may be located near the lower end 64 or at any other desirable location. The feature may be a clip that snap fits around the shunt catheter 72 or any other suitable mechanism for securing the catheter 72.

The shunt catheter 72 is tubular defining a lumen configured to transport excess cerebrospinal fluid out of the brain. The shunt catheter 72 may include a plurality of inlet apertures in fluid communication with the lumen for collecting the fluid. The shunt catheter 72 may be connected with a one-way valve 140 that allows fluid to exit the shunt catheter 72 while preventing backflow into the shunt catheter 72. The outlet side of the one-way valve 140 is connected to an external catheter 142 that transports the fluid away. The shunt one-way valve 140 may be at least partially received within the burr hole 24 adjacent to the riser 56. As discussed above, the minimized footprint of the riser 56 provides space for the one-way valve 140. Additionally, the open guide 68 allows easier insertion and/or removal of the catheter 72.

The device 20 may also be used in conjunction with an EVD. The EVD may include a cerebrospinal fluid (csf) catheter similar to the catheter 72 that is insertable into the slot 36 and the guide 68 of the device 20. The EVD may a way to control csf flow external to the patient.

The open slot of the flange 22 and the open guide of the elongated body 60 cooperate to allow space of other components, e.g., a brain tissue oxygen monitoring sensor, a brain pressure sensor, and/or a pH sensor, which may be placed alongside the csf drainage tubing from the ventricle and also to allow easier replacement should the csf drainage tube become blocked or malfunction thereby requiring replacement.

The intracranial implant sensing device 20 may be provided as a stand-alone component or may be provided as kit or system including a VP shunt (or EVD or ventriculostomy) and/or the data processing and storage system 111.

The implant 20 and the VP shunt 70 (or other intracranial catheter) may be implanted in the patient at the same time or the implant 20 may be implanted first and serves as a guide for later inserting the catheter 72 or the like. The catheter 72 is removable without removal of the implant 20. The open guide 68 of the elongate body 60 may facilitate removal of the catheter 72 by reducing friction therewith. This allows for the device 20 to remain implanted even if revision procedures are needed to replace the catheter 72.

One or more sensing electrodes may be placed on the catheter of the VP shunt or EVD. For example, at least one sensing electrode may be disposed on an outer sidewall of the catheter 72. The electrode may be electrically connected to the wiring harness 104 or include dedicated wiring. In one embodiment, the device 20 includes a connector located on the flange, riser, anchor plate, or elongated body and configured to couple with a connector associated with the catheter electrode. Here, the connector includes an associated wire integrated with the wiring harness 104. The wire may be disposed within the anchor plate, riser, flange, or elongate body similar to the wires 100, 102.

The data collected by the above-described devices and systems may be used to continuously measure high-quality temporally relevant brain data over long periods of time in patients in which EVDs, VP shunts, or the like are, or have been, implanted. The collected data may be used, but is not limited, to diagnosis a current or future brain state, such as a brain state associated with disease, mental illness, seizure, sleep state, response to medication, side effect of medication, level of alertness and mental clarity, cognitive clarity, emotional state, capacity to make decisions, alert status, ability to communicate and work with others, personal preferences, response to traumatic and other therapeutic stimulation, V-P shunt malfunction, among others.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to strength, durability, marketability, appearance, packaging, size, serviceability, weight, manufacturability, ease of assembly, etc. As such, embodiments described as less desirable than other embodiments or prior art implementations with respect to one or more characteristics are not outside the scope of the disclosure and can be desirable for particular applications.

What is claimed is:

1. An intracranial implant sensing device comprising:
a flange receivable within a burr hole formed in a human skull and defining an elongated slot;
an elongate body including an arcuate sidewall defining an open guide for a ventricular catheter, the elongate body being connected to the flange such that a portion of the sidewall is disposed within or at a perimeter of the slot; and
a sensing electrode connected to the sidewall of the elongate body.

2. The intercranial implant of claim 1 further comprising a riser extending upwardly from the flange; and an anchor plate extending radially outward from an upper end of the riser and restable on the human skull.

3. The intracranial implant of claim 1, wherein the flange has a top and an arcuate sidewall depending from the top and extending circumferentially less than 350 degrees to define an open end of the elongated slot.

4. The intracranial implant of claim 2, wherein a lower end of the riser is connected to the arcuate sidewall.

5. The intracranial implant of claim 2, wherein the riser, the anchor plate, and the flange are integrally formed.

6. The intracranial implant of claim 1, wherein the sensing electrode is configured to sense intracranial electroencephalogram signals.

7. The intracranial implant of claim 1, wherein the elongate body includes a connection feature configured to couple with a cerebrospinal fluid catheter.

8. The intracranial implant of claim 1, wherein the sidewall of the elongate body circumferentially extends between 90-270. degrees, inclusive.

9. The intracranial implant of claim 1 further comprising a second sensing electrode connected to the sidewall and longitudinally spaced from the sensing electrode.

10. The intracranial implant of claim 1 further comprising a ground electrode at least partially surrounding the elongate body.

11. The intracranial implant of claim 3, wherein the open side of the guide is circumferentially aligned with the open end of the slot.

12. The intracranial implant of claim 2 further comprising a wire having a first end connected to the sensing electrode and extending through the riser and anchor plate.

13. The intracranial implant of claim 12 further comprising a coil connected to a second end of the wire and configured to inductively couple with a second coil.

14. A medical device comprising:
a sensing device including:
an intracranial implant having a flange receivable within a burr hole formed in a human skull and defining an elongated slot, an anchor plate fixed to the implant such that the anchor plate is spaced above the flange and is configured to rest on the human skull, and a sensor stem joined to the flange and insertable into a brain,
a first electronic device connected to the electrode and implantable subcutaneously, and
a second electronic device receivable by the first electronic device to electrically couple with the first electronic device and receive data collected by the electrode;
a cerebrospinal fluid catheter receivable within the stem; and
at least one sensing electrode disposed on the stem or the catheter.

15. The medical device of claim 14, wherein the stem has a curved sidewall that defines an open guide configured to receive the catheter.

16. The medical device of claim 14, wherein the elongated slot is an open slot having an entrance defined between opposing walls of the flange, wherein the stem has a partial sidewall having an open side that is aligned with the entrance of the open slot, wherein the stem includes a first connection feature configured to secure the catheter in the open slot.

17. The medical device of claim 14, wherein the implant further has a riser connected between the anchor plate and the flange.

18. The medical device of claim 14, wherein the at least one sensing electrode is at least two sensing electrodes.

19. The medical device of claim 14, wherein the flange is horseshoe shaped.

20. The medical device of claim 19, wherein the sensing device further includes a horseshoe-shaped ground electrode disposed on a bottom of the flange.

21. A system for collecting and sending intracranial electroencephalogram data, the system comprising:
an implant including:
a burr-hole flange defining a first open slot,
an anchor plate located above a top of the flange,
a riser connected between the flange and the anchor plate,
an elongate body extending downwardly from the flange, the elongate body having a partial arcuate sidewall defining an open guide, and
a sensing electrode connected to the elongate body;
a first device configured to receive data from the electrode;
a second device electrically coupled to the first device; and
a storage device electrically coupled to the second device and configured to store the data.
